(19) United States
(12) Patent Application Publication
(10) Pub. No.: US 2022/0117896 A1
(43) Pub. Date: Apr. 21, 2022

(54) Alginate Beads and Methods of Manufacture and Use Thereof

(71) Applicant: CLEARH2O, INC., Portland, ME (US)

(72) Inventor: Jay PALMER, Brunswick, ME (US)

(21) Appl. No.: 17/046,580

(22) PCT Filed: Apr. 16, 2019
(86) PCT No.: PCT/US2019/027588

§ 371 Date: Oct. 9, 2020

Related U.S. Application Data
(60) Provisional application No. 62/658,831, filed on Apr. 17, 2018.

(51) Int. Cl. A61K 9/16; A23K 20/163; A23K 50/75; A23K 20/195

(52) U.S. Cl. CPC A61K 9/1652; A23K 20/163; C08L 5/04; A23K 20/195; A23K 50/75

(57) ABSTRACT

Alginate beads and methods of producing said beads, the beads being able to contain hydration, nutritional additives, medications, colorants, sweeteners and/or flavorings, and that are particularly advantageous for use as top dressing for poultry feed.

### FIELD OF THE INVENTION

This disclosure relates to alginate beads and, particularly, to alginate beads used as top dressing for poultry feed.

### BACKGROUND

In the agriculture and laboratory animal research industries it is often necessary to provide animals such as rodents, poultry, rabbits, swine, cattle, and others with vaccines, medications, and nutritional additives as well as additional hydration. In the poultry industry in particular, once poultry chicks reach a certain age they are regularly provided with feed consumed orally in order to meet their nutritional needs. However, sometimes chicks need more than just nutrition.

In order to provide vaccines and nutritional additives (e.g., probiotics) to chicks, top dressings containing the desired substance(s) may be added on top of the chick's feed. Conventional top dressings are dry or powered substances that are either poured or sprinkled on top of the feed or mixed into the feed. However, additives delivered in this conventional manner are not always easy to consume and may not be uniformly distributed throughout the feed. Additionally, dry or powered top dressings provide no hydration to the chicks.

### SUMMARY

Alginate beads designed for use as top feed for poultry are disclosed herein. The disclosed alginate beads have a relatively high water content (e.g., at least 50%) and can thereby provide the poultry with an appreciable amount of hydration. In addition to hydration, the disclosed alginate beads may also contain, in some embodiments, one or more additives, such as vaccines, probiotics, nutrients, colorant, and/or other desired substances. The disclosed alginate beads may, in some cases, serve as a top dressing (for poultry or other types of foul and/or livestock) that is easily consumable, contains the desired additives, and provides hydration.

In another aspect, methods of producing alginate beads are disclosed. For example, in some embodiments, the disclosed alginate beads may be produced by forming an alginate solution and an ionic solution. The alginate solution may include, in some embodiments, an alginate copolymer, water, and one or more additives (e.g., vaccines, probiotics, nutrients, colorant, and/or other desired substances). The ionic solution may include, in some embodiments, water and a divalent cation (e.g., calcium chloride). Droplets of the alginate solution may then be introduced into the ionic solution. Upon contact with the ionic solution, the alginate solution forms alginate beads, which may then be separated from the ionic solution. The alginate beads may then optionally be further processed (e.g., by rinsing with water, drying, and/or packaging for transport).

### BRIEF DESCRIPTION OF THE DRAWINGS

FIG. 1 shows a photograph of exemplary alginate beads, in accordance with some embodiments of the subject disclosure.

FIG. 2 shows a photograph of exemplary alginate beads, in accordance with some embodiments of the subject disclosure.

FIG. 3 shows a photograph of exemplary alginate beads, in accordance with some embodiments of the subject disclosure.

FIG. 4 shows a photograph of exemplary alginate beads provided to poultry, in accordance with some embodiments of the subject disclosure.

FIG. 5 shows a flow diagram of an exemplary method of producing alginate beads, in accordance with some embodiments of the subject disclosure.

### DETAILED DESCRIPTION

The present technology will now be described more fully in detail and with reference to the accompanying drawings, in which select embodiments of the present disclosure are shown. This disclosure should not, however, be construed as limited to the particular embodiments set forth herein or illustrated in the accompanying drawings; rather, the embodiments discussed herein are provided so that this disclosure will fully convey the scope of the presently disclosed technology to those skilled in the art.

Alginate beads are disclosed herein, in addition to methods of manufacture and use thereof. The disclosed alginate beads may be provided in gel form, and more specifically, as hydrogels, which contain a relatively high amount of water. As described in detail below, the disclosed alginate beads can be particularly useful as top dressing for poultry and, in some such embodiments, the alginate beads may deliver hydration and one or more desired additives, such as medication and/or nutritional supplementation to the poultry. It is important to note that while the present disclosure discusses alginate beads designed for use with poultry (e.g., poultry chicks, or simply “chicks”) in detail, it is understood that the present disclosure is not intended to be so limited. For example, the disclosed alginate beads may be used with other types of poultry, such as turkey, duck, geese, pheasant, quail, peacock, grouse, and/or partridge, or with any other suitable livestock animals, such as sheep, goats, llamas, donkeys, pigs, cows, and/or horses. Additionally, in some cases, the disclosed alginate beads may be administered to laboratory research animals, such as rodents and/or rabbits.

Alginate, also referred to sometimes as “alginic acid” or “algin,” is an anionic polysaccharide having the following chemical formula:

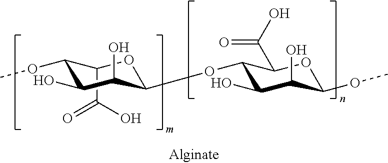

Alginate is extracted from brown algae and is commercially available in the form of sodium alginate (NaC₆H₇O₆). As is evident from its chemical formula, alginate is a copolymer with homopolymeric blocks of (1-4)-linked β-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks), or alternating M and G-residues (MG-blocks). The disclosed alginate beads may include any type of alginate copolymer, such as M-block, G-block, and/or MG-block alginate copolymers.

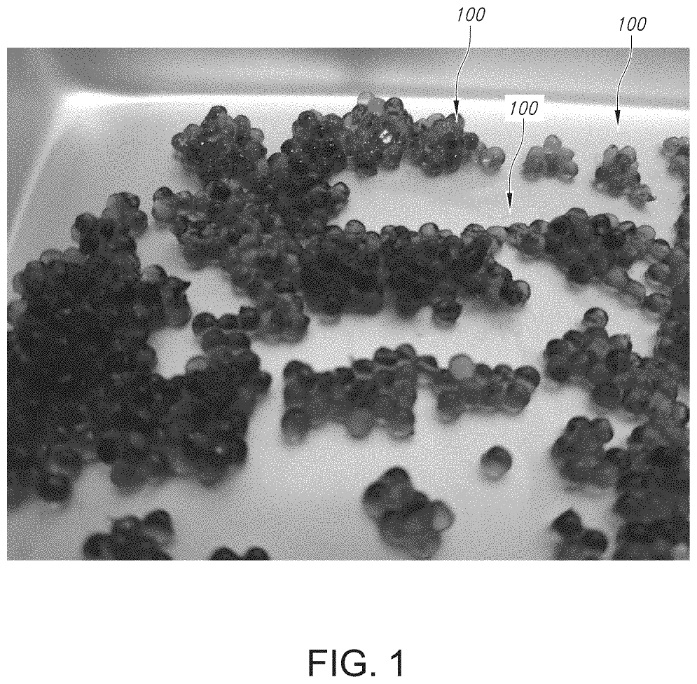

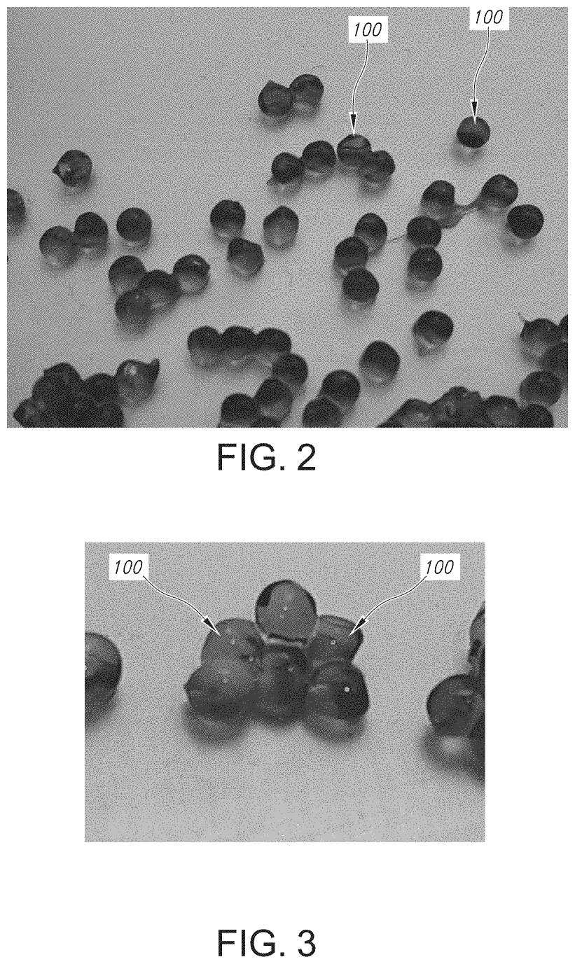

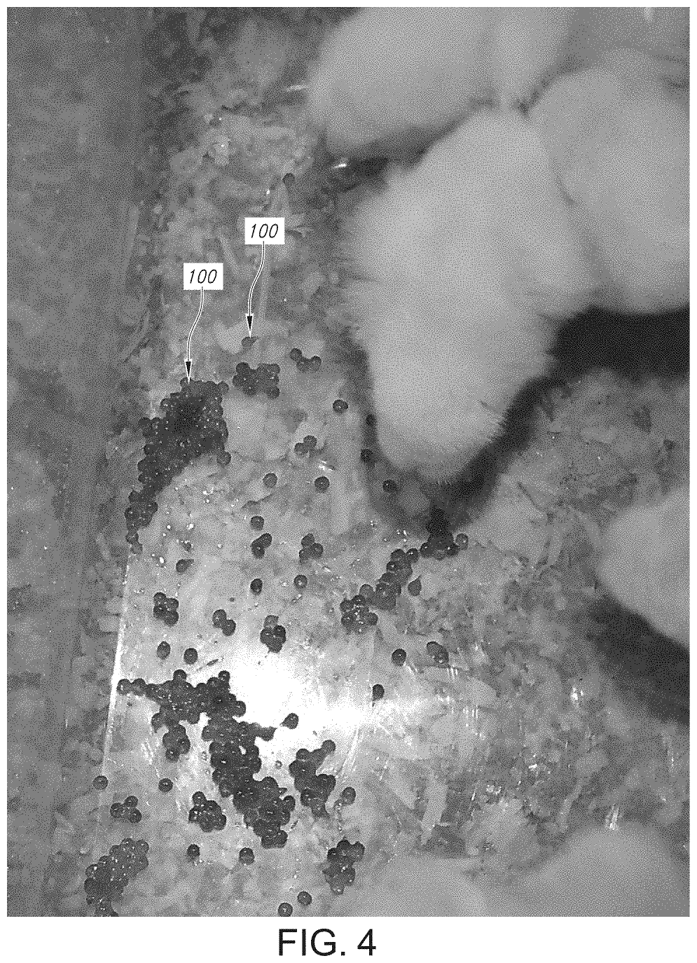

Example alginate beads are shown in FIGS. 1-4. In particular, FIG. 1 shows a plurality of alginate beads 100, FIG. 2 shows a plurality of alginate beads 100 with higher magnification, and FIG. 3 shows a plurality of alginate beads 100 with even higher magnification. FIG. 4 shows a plurality of alginate beads 100 being administered to a group of chicks. As shown In FIGS. 1-4, the alginate beads may be a firm or semi-firm gel beads and each bead may have an approximately spherical shape.

The disclosed alginate beads 100 can be designed to have any suitable composition. For example, in some embodiments, the alginate beads may contain water, an alginate copolymer, and one or more additives. The one or more additives present in the disclosed alginate beads may be selected based on the purpose of the product. For example, if the alginate beads are to be administered to young poultry (i.e., chicks less than 10 days old), the alginate beads may include one or more nutritional additives (e.g., carbohydrates, proteins, vitamins, fats, probiotics, prebiotics, minerals), medical additives (e.g., vaccines, antibiotics), and/or colorant. In select embodiments, the disclosed alginate beads include at least one additive, which is either a nutritional additive or a medicinal additive. In these and other embodiments, the alginate beads may include at least one nutritional additive and at least one medicinal additive. If desired, sweetener, flavoring, and/or fragrance may also be included in the alginate beads. As will be understood, numerous configurations and variations of alginate bead composition are possible, and the composition of an alginate bead may vary based on the intended use of the alginate bead. Some example alginate bead additives are provided below for exemplary purposes and are not intended to limit the scope of the subject disclosure.

In some embodiments, the disclosed alginate beads 100 may include one or more nutritional additives in a total weight percent of at least 5%. In these and other embodiments, the alginate beads may include one or more carbohydrates. If present, the carbohydrate content of the alginate bead may be between 1% and 30% (by weight or by volume), such as between 5% and 25%, or between 10% and 20%. Example carbohydrates that may be used include both corn-based, wheat-based, or other types of carbohydrates. For alginate beads that include proteins and/or amino acids, the proteins and/or amino acids may be derived from vegetables and/or fish. Proteins and/or amino acids may be present in a weight or volume percent of between 0% and 20%, such as 5%-15%, or 7%-12%. Other additives that may be present in the disclosed alginate beads include vitamins (e.g., A, C, D, E, K), minerals, fats (corn oil, soy oil, canola oil, linoleic acid), vaccines, antibiotics, colorant (green, blue, red, or other), fragrance, and/or sweetener (e.g., cane sugar or a corn-based sweetener). These additives may be present in any amount, such as less than 5% by weight or by volume.

The water content of the alginate beads may be at least 50%, or within the range of 50%-95%, 60%-85%, or 70%-80% by weight or by volume, in some embodiments. The total concentration of alginate copolymer in the disclosed alginate beads may be between 0.1% and 10%, such as 1%-5% by weight or by volume.

The alginate beads may have any desired dimensions. In some embodiments, the alginate beads are approximately spherical and have an approximate diameter of between 1 millimeter (mm) and 10 mm, between 2 mm and 6 mm, or between 1 mm and 5 mm.

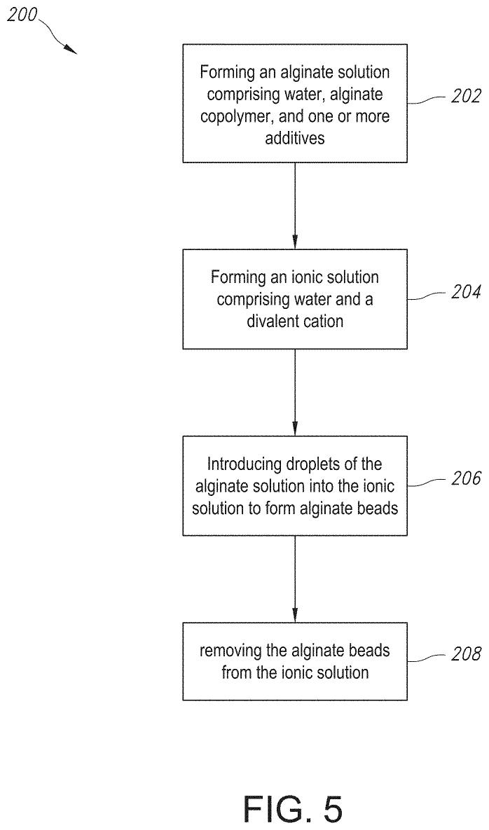

The disclosed alginate beads may be produced by any suitable method. An example method of producing alginate beads is shown in FIG. 5. As shown in FIG. 5, alginate beads may be produced by method 200, which includes forming 202 an alginate solution comprising water, an alginate copolymer, and one or more additives. The alginate solution may contain any additives or combination of additives discussed previously with respect to alginate beads 100. In some embodiments, the alginate solution may contain between 50%-80% water, 0%-30% carbohydrate, and 0%-20% protein by weight (g/g) or by volume (g/mL). In select embodiments, the alginate solution comprises between 65%-75% water, 15%-25% carbohydrate, and 5%-15% protein (g/g or g/mL). In one particular embodiment, the alginate solution includes approximately 70% water, 20% carbohydrate, and 10% protein (g/mL). The amount of alginate copolymer (e.g., sodium alginate, in some embodiments) present in the alginate solution may be between 0.01-5% (g/g or g/mL). In select embodiments, the alginate solution includes between 1 and 4% alginate copolymer (g/g or g/mL). The compounds present in the alginate solution may be dissolved in water by mixing. If required, heat may also be administered to the alginate solution to facilitate dissolution.

Method 200 of FIG. 5 continues with forming 204 an ionic solution comprising water and a divalent cation. In some embodiments, the divalent cation is calcium and calcium chloride is used to form 204 the ionic solution. However, other divalent cations may also be used, as appropriate. In select embodiments, the ionic solution includes between 1% and 10%, or between 2 and 8% calcium chloride (g/g or g/mL). After the divalent cation is added to the water of the ionic solution, the solution may be thoroughly mixed (with or without heat) until the divalent cation has completely dissolved.

Method 200 of FIG. 5 continues with introducing 206 droplets of the alginate solution into the ionic solution. In some embodiments, droplets of the alginate solution may be formed by slowly dripping the alginate solution into the ionic solution or, in other embodiments, a pipette or dropper may be used to form droplets of the alginate solution. The size of the alginate solution droplets may be directly proportional to the diameter of the resulting alginate beads. Accordingly, the droplet size of the alginate solution may be selected based on desired dimensions of the alginate beads. As droplets of the alginate solution are introduced into the ionic solution, the ionic solution may be kept at, below, or above room temperature. As the droplets of the alginate solution encounter the ionic solution, the alginate solution crosslinks with the divalent cation and forms a firm, sticky alginate bead that includes the additive(s) present in the alginate solution.

Method 200 of FIG. 5 continues with removing 208 the alginate beads from the ionic solution. The alginate beads may remain in the ionic solution for any predetermined period of time prior to removal. For example, in some embodiments, the alginate beads may be removed from the ionic solution within 1 hour, within 45 minutes, within 30 minutes, within 20 minutes, within 10 minutes, or within 5 minutes. The alginate beads may be removed 208 from the ionic solution by any appropriate technique, including by sifting, size-based filtration, evaporation of the ionic solution, or another suitable process. After removal from the ionic solution, the alginate beads may optionally be processed. Additional processing may include, in some embodiments, rinsing with water, drying (at least partially), and/or packaging for consumer use.

After production, the alginate beads may be provided to poultry or other animals. The disclosed alginate beads may be durable and resilient enough to be scooped, sprinkled, or shoveled onto the top of conventional feed. Additionally, the disclosed alginate beads may also be slightly sticky, easily adhering to one another and to feed, primarily to the top surface of the feed, thereby making consumption by the chicks far more likely than with conventional top dressings. Further, the alginate beads may maintain their structure and shape, remaining available for the chicks to consume over a period of up to 96 hours. The spherical and uniform shape and size of the beads may allow the chicks to consume individual “bites,” making the alginate beads a very precise and accurate dosing system. This is in contrast to other high moisture gels that are offered to chicks as oatmeal like mush or slabs of gel. The disclosed alginate beads thus offer a distinct and unique advantage, allowing for optimum consumption and enabling and stimulating natural feeding behavior.

It is understood that the embodiments described herein are merely illustrative. Variations in the formulation of the alginate beads and related methods may be contemplated by one skilled in the art without limiting the intended scope of the present disclosure, and as defined by the following claims.

What is claimed is:

1. An alginate bead comprising:
an alginate copolymer;
at least 50% water by weight; and
at least one additive, which is either a nutritional additive or a medicinal additive.

2. The alginate bead of claim 1, wherein the alginate copolymer is present in a weight percent of between 0.1% and 10%.

3. The alginate bead of claim 2, wherein the alginate copolymer is present in a weight percent of between 1% and 5%.

4. The alginate bead of claim 1, wherein the at least one additive is a nutritional additive selected from the group consisting of: carbohydrates, proteins, vitamins, fats, probiotics, prebiotics, and minerals.

5. The alginate bead of claim 4, wherein the nutritional additive is present in a weight percent of at least 5%.

6. The alginate bead of claim 1, wherein the at least one additive is a medicinal additive that is a vaccine or an antibiotic.

7. The alginate bead of claim 1, wherein the alginate bead comprises at least one nutritional additive and at least one medicinal additive.

8. The alginate bead of claim 1 further comprising a sweetener, flavoring, or fragrance.

9. The alginate bead of claim 1 further comprising a colorant.

10. A method of producing an alginate bead, the method comprising:
forming an alginate solution comprising sodium alginate, water, and at least one additive, which is either a nutritional additive or a medicinal additive;
forming an ionic solution comprising water and a divalent cation;
introducing droplets of the alginate solution into the ionic solution to form alginate beads; and
removing the alginate beads from the ionic solution.

11. The method of claim 10, wherein the divalent cation is calcium and calcium chloride is used to form the ionic solution.

12. The method of claim 10, wherein the at least one additive is a nutritional additive selected from the group consisting of: carbohydrates, proteins, vitamins, fats, probiotics, prebiotics, and minerals.

13. The method of claim 10, wherein the at least one additive is a medicinal additive that is a vaccine or an antibiotic.

14. The method of claim 10, wherein the alginate solution comprises at least one nutritional additive and at least one medicinal additive.

15. The method of claim 10, wherein the alginate solution further comprises a colorant.
